(19) United States
(12) Patent Application Publication
(10) Pub. No.: US 2020/0315745 A1
(43) Pub. Date: Oct. 8, 2020

(54) VARIABLE-FREQUENCY ORAL VIBRATION SYSTEMS AND METHODS

(71) Applicant: Advanced Orthodontics and Education Association, LLC, Ossining, NY (US)

(72) Inventors: Bryce A. WAY, San Jose, CA (US); Richard JOHNSON, Briarcliff Manor, NY (US)

(73) Assignee: Advanced Orthodontics and Education Association, LLC, Ossining, NY (US)

(21) Appl. No.: 16/787,445

(22) Filed: Feb. 11, 2020

Related U.S. Application Data
(63) Continuation-in-part of application No. 16/102,264, filed on Aug. 13, 2018.
Application No. 16/102,264 is a continuation of application No. 13/828,692, filed on Mar. 14, 2013, now Pat. No. 10,085,822.
Application No. 13/828,692 is a continuation-in-part of application No. 16/142,461, filed on Sep. 26, 2018.
(60) Provisional application No. 61/624,100, filed on Apr. 13, 2012.

(51) Int. Cl. A61C 7/00; A61C 7/08

(52) U.S. Cl. CPC A61C 7/008; A61F 2002/2864; A61C 7/08

(57) ABSTRACT

A dental device includes a mouthpiece configured to sit against occlusal surfaces of a patient's teeth and a motor connected to the mouthpiece. The motor is configured to vibrate the mouthpiece at a frequency between 60 and 120 Hz and an acceleration between 0.03G and 0.06G such that the mouthpiece places an axial vibratory force on the occlusal surfaces.

### CROSS-REFERENCE TO RELATED APPLICATIONS

This application is a continuation-in-part of pending co-assigned U.S. patent application Ser. No. 16/102,264, filed Aug. 13, 2018, which is a continuation of U.S. patent application Ser. No. 13/828,692, filed Mar. 14, 2013, now U.S. Pat. No. 10,085,822 issued on Oct. 2, 2018, which claims priority to U.S. Provisional Application No. 61/624,100, filed Apr. 13, 2012. This application is also a continuation-in-part of pending co-assigned U.S. patent application Ser. No. 16/142,461 filed Sep. 26, 2018. The entireties of each of the above applications are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Orthodontic treatment continues to grow in popularity among both teens and adults. While social stigmas associated with orthodontic treatment are in decline, many are still hesitant to consider treatment. The length of treatment time, and fear of pain associated with treatment are the most prevalent concerns cited as the barriers to treatment acceptance. Studies have demonstrated that 58.3% of the subjects cited orthodontic pain as their primary complaint, followed closely by treatment duration. One factor that contributes to the pain and discomfort felt by the patients is poor aligner seating. When the aligners are not adequately seated, the aligner tray can lose its grip around the patient's teeth, which results in improper distribution of forces on the teeth. Therefore, the patient's teeth can move in an unexpected or non-advantageous manner, thereby producing pain and discomfort for the patients. As competition for new patients continues to increase, successful orthodontic practices continue to seek ways to differentiate their services, while addressing these cited concerns of potential and existing patients.

Vibration in conjunction with orthodontic forces has been studied in various frequencies and force levels with mixed results (Woodhouse 2015 and Ottoson 1981). It appears that frequency and force appear to correlate with the therapeutic responses associated with vibration therapy (Lala 2016). Previous literature and studies have demonstrated that vibration at low frequency was not effective at reducing pain originating from teeth (Woodhouse 2015 and Lala 2016), where vibration at high frequency was (Ottoson 1981 and Lala 2016). A possible mechanism is the “gate control” theory, which suggests that pain can be reduced by simultaneous activation of nerve fibers that conduct non-noxious stimuli. Another possibility is that vibration may help relieve compression of the periodontal ligament (PDL), thus promoting normalized circulation (Long 2016). In addition, high frequency vibration may improve seating of the aligners, thereby eliminating unplanned and unwanted teeth movement, allowing better tracking of teeth movement, and ultimately reducing pain and discomfort.

Use of nonsteroidal anti-inflammatory drugs (NSAIDs) to manage pain conjunction with orthodontic tooth movement has been shown to decrease prostaglandin synthesis leading to a decrease in the inflammatory bone resorption process and may negatively impact tooth movement. Therefore, efforts to find ways to increase compliance and manage pain as it relates to patient treatment satisfaction, as well as ways to provide more efficient treatment continue, along with efforts to address perceived pain for patients reluctant to accept treatment.

In addition to reducing pain and discomfort, mechanical vibration may enhance musculoskeletal properties. For example, some studies suggest that low-intensity mechanical vibrations may stimulate bone formation or mitigate the degradation of the intervertebral disc in rats. However, the biomolecular mechanisms for such enhancement effects have not yet been elucidated. Some studies suggested that mechanical vibration may enhance differentiation of human bone marrow mesenchymal stem cells or periodontal ligament stem cells. But discrepancies and unpredictability exist in literature as to the effects of mechanical vibration on cell proliferation. For example, previous studies have demonstrated no effects or either increased or decreased proliferation after cyclic vibration treatment (Zhang 2012).

Other efforts to accelerate tooth movement during orthodontic treatment have included application of low-frequency vibration to the teeth while the teeth are being treated with an orthodontic appliance. Woodhouse, N. R. et al. (“Supplemental Vibrational Force During Orthodontic Alignment: A Randomized Trial”), Journal of Dental Research 94(5): 682-689 (2015), which is incorporated by reference herein, investigated the effects of low frequency vibration on the rate of tooth movement in patients with fixed appliances (i.e., braces). Subjects with fixed appliances were treated with the commercially available dental device AcceleDent™, developed by OrthoAccel® Technologies, Inc., which provides a vibrational frequency of 30 Hz and a force of 0.2 N to the teeth being treated with the fixed appliances. Subjects were treated with AcceleDent™ daily for 20 minutes per day until the teeth reached final alignment, and were compared with controls with fixed appliances who had not been treated with AcceleDent™. Woodhouse found no evidence that 30 Hz treatment with AcceleDent™ significantly increased the rate of tooth movement or reduced the amount of time required for the teeth to achieve final alignment, when used in conjunction with fixed appliances.

It is recently hypothesized that mechanical vibration may promote periodontal regeneration and periodontal tissue remodeling during and following orthodontic tooth movement. However, variables of mechanical vibration to be used for modulating bone biology so as to effectively accelerate orthodontic tooth movement remain to be determined.

It has been shown that high frequency forces, even at low magnitude, are able to stimulate bone formation and increase bone mass. The dental devices described herein are intended to provide the appropriate force to grow and strengthen bone in the mouth.

It would be advantageous to have a device and method for delivering vibration to the user's dentition in order to manage pain, enhance musculoskeletal properties, accelerate tooth movement, and improve seating of aligners. For example, when used in conjunction with orthodontic treatments, such as bracket-and-wire braces or aligners, the device could successfully reduce oral pain or discomfort of any etiology as well as the duration of treatment by delivering high frequency vibration to the dentition of the user.

### SUMMARY OF THE DISCLOSURE

The present disclosure relates generally to dental devices. More specifically, the present disclosure relates to vibratory dental devices used for modifying bone density in the mouth, such as increasing bone density for orthodontic retention. The devices of the present disclosure can also be used for orthodontic acceleration and/or for the seating of orthodontic aligners onto the dentition of a user. The embodiments of the present disclosure further include devices, systems, and methods for accelerating tooth movement in aligner treatment with high-frequency vibration. Advantageously, the exemplary embodiments provide a method of accelerating tooth movement while maintaining tracking.

In general, in one embodiment, a dental device includes a mouthpiece configured to sit against occlusal surfaces of a patient's teeth. The dental device further includes a vibrator connected to the mouthpiece. The vibrator is configured to deliver a vibratory waveform to the mouthpiece. In some embodiments, the waveform can be oscillatory. In some embodiments, the vibratory waveform can be substantially continuously variable. In some embodiments, the vibratory frequency can be continuously variable between an upper and lower threshold, for example between 60 Hz and 150 Hz. In some embodiments the vibratory waveform can be delivered at an acceleration between 0.030 G and 0.200 G. The device can further include, in some aspects, a vibration unit. The vibration unit can be configured to deliver vibration to the dentition of the user. The vibration unit can be removably coupled to the plurality of pads, the mouthpiece body, or both. The vibration unit, in some aspects, can include a power source and a motor for adjusting a frequency or g-force of vibration. The vibration unit can deliver vibration at a frequency between about 30 Hz and about 200 Hz. The vibration frequency can be, for example, from about 80 Hz to about 120 Hz, from about 110 Hz to about 120 Hz, from about 100 Hz to about 110 Hz, from about 90 Hz to about 100 Hz, or from about 80 Hz to about 90 Hz. It is contemplated that in other embodiments, the frequency could be any value within the range of about 30 Hz to about 200 Hz, and that the vibration frequency could be adjusted during a treatment period. In one exemplary embodiment, the vibration frequency is about 100 Hz. In other aspects, the vibration unit can deliver vibration at a g-force between about 0.01 G and about 0.5 G. In some embodiments, the vibration unit can deliver vibration, for example, at a g-force between about 0.03 G and about 0.2 G. The g-force of the vibration can be adjusted.

This and other embodiments can include one or more of the following features. The frequency can be between 100 Hz and 120 Hz or between 110 Hz and 130 Hz. The acceleration can be between 0.05 G and 0.06 G. The vibrator can vary the frequency continuously, for example in a sinusoidal fashion, increasing and then decreasing between a maximum and a minimum threshold. The maximum and minimum frequencies, as well as the sweep rate between them can be made programmable. The vibrator can also be configured to oscillate between discrete frequencies and accelerations. For example, the vibrator can be configured to oscillate between four specific settings. The four specific settings can be 60 hz at 0.035G, 60 hz at 0.06G, 120 hz at 0.035 G, and 120 hz at 0.06 G. The mouthpiece can include a biteplate configured to sit against occlusal surfaces of a patient's teeth and an extension configured to connect to a base. The mouthpiece can have a U-shape so as to extend over all of a patient's teeth or can be configured to contact only selected teeth. The dental device can further include a controller configured to adjust the vibrator settings, which in some embodiments can be based upon a detected-vibration feedback loop.

In an illustrative embodiment, the controller can be a surface-mount low power chip such as the BGM121/BGM123 Blue Gecko BLUETOOTH SiP Module available from Silicon Labs of Austin, Tex., U.S.A. The controller can advantageously integrate a BLUETOOTH stack, such as BLUETOOTH Low Energy, and can also run end-user applications on-board for motor control. Alternatively, the chip can be used as a network co-processor over a host interface.

In general, in one embodiment, a method of growing bone, accelerating orthodontic treatment or seating oral appliances including aligners includes contacting a mouthpiece over occlusal surfaces of a patient's teeth, vibrating the mouthpiece at a substantially constantly-varying frequency centered at or about 120 Hz, for example between 110 Hz and 130 Hz. The acceleration can be between 0.030 G and 0.20 G, and treatment can include repeating the placing and vibrating steps for 10 minutes per day or less, or for 5 minutes per day or less for 180 days or less to achieve periodontal ligament growth around the teeth or accelerated orthodontic treatment. The device can also be used to seat aligners onto a user's teeth to ensure optimal placement and contact recommended for the best results in aligner therapy.

This and other embodiments can include one or more of the following features. The frequency can be varied between 100 Hz and 140 Hz. The acceleration can be constant or can vary with changes in frequency. Repeating the placing and vibrating steps for less than 5 minutes per day can include repeating the placing and vibrating steps for less than 2 minutes per day. Repeating the placing and vibrating steps for less than 180 days can include repeating the placing and vibrating steps for less than 120 days. The method can further include placing a retainer over the occlusal surfaces of the teeth between repetitions. As used herein, “daily” or “per day” can alternatively mean each and every day, or only those days where treatment is administered, unless context makes clear that one or the other alternative meaning is intended.

In general, in one embodiment, a dental device includes a mouthpiece configured to sit against occlusal surfaces of a patient's teeth and a motor connected to the mouthpiece. The vibrator is configured to vibrate the mouthpiece at a frequency between 60 Hz and 130 Hz and an acceleration between 0.035 G and 0.100 G such that the mouthpiece places an axial vibratory force on the occlusal surfaces. Further, the dental device weighs less than 50 grams.

This and other embodiments can include one or more of the following features. The motor can require less than 2 volts to vibrate the mouthpiece. The frequency can be between 100 Hz and 120 Hz. The acceleration can be between 0.05 G and 0.06 G. The motor can be configured to oscillate between frequencies and accelerations. The motor can be configured to oscillate between four specific settings. The four specific settings can be 60 hz at 0.035G, 60 hz at 0.06G, 120 hz at 0.035 G, and 120 hz at 0.06 G. The mouthpiece can be customized to fit the patient's teeth. The mouthpiece can include a biteplate configured to sit against occlusal surfaces of a patient's teeth and an extension configured to connect to a base. The motor can be a counterweighted motor that is substantially in-line with a longitudinal axis of the extension. The motor can be a pancake motor. The mouthpiece can have a U-shape so as to extend over all of a patient's teeth. The mouthpiece can be configured to extend only over a patient's social six teeth. The mouthpiece can be configured to extend only over a patient's molars. The dental device can further include a sensor configured to detect the vibration proximate to the occlusal surfaces of the patient's teeth. The dental device can further include a controller configured to adjust the motor settings based upon the detected vibration.

In general, in one embodiment, a dental device includes a mouthpiece configured to sit against occlusal surfaces of a patient's teeth. The dental device further includes a motor connected to the mouthpiece. The motor is configured to vibrate the mouthpiece at a frequency between 60 Hz and 130 Hz and an acceleration between 0.035 G and 0.100 G such that the mouthpiece places an axial vibratory force on the occlusal surfaces. The dental device further includes a sensor configured to detect the vibration proximate to the occlusal surfaces of the patient's teeth.

This and other embodiments can include one or more of the following features. The dental device can further include a controller configured to adjust the motor settings based upon the detected vibration. The sensor can be a piezoelectric sensor. The frequency can be between 100 Hz and 120 Hz. The acceleration can be between 0.05 G and 0.06 G. The motor can be configured to oscillate between frequencies and accelerations. The motor can be configured to oscillate between four specific settings. The four specific settings can be 60 hz at 0.035G, 60 hz at 0.06G, 120 hz at 0.035 G, and 120 hz at 0.06 G. The mouthpiece can be customized to fit the patient's teeth. The mouthpiece can include a biteplate configured to sit against occlusal surfaces of a patient's teeth and an extension configured to connect to a base. The motor can be a counterweighted motor that is substantially in-line with a longitudinal axis of the extension. The motor can be a pancake motor. The mouthpiece can have a U-shape so as to extend over all of a patient's teeth. The mouthpiece can be configured to extend only over a patient's social six teeth. The mouthpiece can be configured to extend only over a patient's molars. The dental device can further include a sensor configured to detect the vibration proximate to the occlusal surfaces of the patient's teeth. The dental device can further include a controller configured to adjust the motor settings based upon the detected vibration.

Methods of using these devices to grow bone, accelerate orthodontic treatment and to seat aligners are also described herein.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows an exemplary dental device having a mouthpiece and base as described herein. FIG. 1B shows the mouthpiece of FIG. 1A disconnected from the base. FIG. 1C shows an exploded view of the mouthpiece and base of FIG. 1A.

FIG. 2 shows vibration of the dental device of FIG. 1.

FIG. 3A shows an exemplary mouthpiece of a dental device having a motor in the mouthpiece positioned inline with the mouthpiece extension. FIG. 3B is an exploded view of the mouthpiece of FIG. 3A. FIG. 3C shows placement of the mouthpiece of FIG. 3A in a patient's mouth.

FIG. 3D is a flowchart for a feedback loop used to adjust the frequency or acceleration of vibration of a dental device as described herein.

FIG. 4A shows an alternative exemplary mouthpiece of a dental device having a motor in the mouthpiece positioned horizontal to the mouthpiece extension and inside the biteplate of the mouthpiece. FIG. 4B is an exploded view of the mouthpiece of FIG. 4A. FIG. 4C shows placement of the mouthpiece of FIG. 4A in a patient's mouth.

FIG. 5A shows an alternative exemplary mouthpiece portion of a dental device having a motor in the mouthpiece positioned horizontal to the mouthpiece extension and outside the biteplate of the mouthpiece. FIG. 5B is an exploded view of the mouthpiece of FIG. 5A. FIG. 5C shows placement of the mouthpiece of FIG. 5A in a patient's mouth.

FIG. 6 is an exploded view of an exemplary base of a dental device described herein.

FIG. 7A shows an exemplary biteplate having raised dimples. FIG. 7B is a cross-section of the biteplate of FIG. 7A.

FIG. 8 shows a biteplate and separable mouthguard of an exemplary mouthpiece as described herein.

FIG. 9 shows an exemplary oven for forming a mouthguard as described herein.

FIG. 10 shows an alternative exemplary oven for forming a mouthguard as described herein.

FIG. 11 shows an exemplary mouthguard having vacuum tubes for forming the mouthguard to a patient's teeth.

FIG. 12A shows an alternative embodiment of a dental device as described herein. FIG. 12B is another view of the mouthpiece of FIG. 12A. FIGS. 12C and 12D show the motor placement in the dental device of FIG. 12A.

FIGS. 13A-F show an alternative embodiment of a mouthpiece as described herein.

FIGS. 14A-14D show an alternative embodiment of a dental device as described herein.

FIGS. 15A-15B show an exemplary charging station for a dental device as described herein.

FIGS. 16A-16D show an alternative exemplary charging station for a dental device as described herein.

FIGS. 17A-17D show an alternative exemplary charging station for a dental device as described herein.

FIG. 18 shows an exemplary connection system between a mouthpiece and a base for a dental device as described herein.

FIG. 19 shows an alternative exemplary connection system between a mouthpiece and a base for a dental device as described herein.

FIG. 20 shows an alternative exemplary connection system between a mouthpiece and a base for a dental device as described herein.

FIG. 21A shows an exploded view of an exemplary vibrating dental device as described herein. FIG. 21B is another view of the device of FIG. 21B. FIGS. 21C-21D show use of the dental device of FIG. 21A.

FIG. 22 shows an exploded view of an alternative exemplary vibrating dental device as described herein.

FIG. 23A shows a base extension having a pancake motor therein. FIG. 23B shows an exemplary pancake motor.

FIG. 24A shows a side-view of a crescent-shaped biteplate for a dental device as described herein. FIG. 24B shows a front view of the crescent-shaped biteplate of FIG. 24A. FIG. 24C shows exemplary use a device having the crescent-shaped biteplate of FIG. 24A.

FIG. 25A shows a side-view double-hammer-shaped biteplate for a dental device as described herein. FIG. 25B shows a front view of the double-hammer-shaped biteplate of FIG. 25A. FIG. 25C shows exemplary use of a device having the double-hammer-shaped biteplate of FIG. 25A.

FIG. 26A shows a side view of an elongated biteplate for a dental device as described herein. FIG. 26B shows a front view of the elongated biteplate of FIG. 26A.

FIG. 26C shows exemplary use of a device having the elongated biteplate of FIG. 26A.

FIGS. 27A-C show front, side, and back views, respectively, of an exemplary base for a dental device as described herein.

FIG. 28 shows exemplary use of a device having the base of FIGS. 27A-C.

FIGS. 29A-D show various exemplary continuously-variable frequency vibration waveforms as described herein.

### DETAILED DESCRIPTION

Described herein are dental devices. The dental devices have or include a mouthpiece with a biteplate configured to sit over all or a portion of the occlusal surfaces of a patient's teeth. The dental devices can be configured to vibrate at a frequency between 60 and 140 HZ and an acceleration between 0.03G and 0.2G such that the mouthpieces places a vibratory force on the occlusal surfaces of the patient's teeth, thereby enhancing tooth growth, accelerating orthodontics, seating aligners, or any combination of these. The seating of aligners can be particularly advantageous in recapturing non-compliant patients that fail to wear their aligners during treatment, leading to difficult fitment. The device can also be used to help reduce the pain accompanying orthodontic procedures, or oral surgery, as described in U.S. Published Patent Application No. 2018-0078337. Other benefits from the oral vibratory waveforms disclosed herein include increasing the proliferation of cells in the vicinity of the periodontal ligament that participate in bone formation or orthodontic tooth movement as described in commonly assigned U.S. patent application Ser. No. 15/875,779 filed on Jan. 19, 2018; decreasing root resorption due to orthodontic forces as described in commonly assigned U.S. patent application Ser. No. 16/139,268 filed on Sep. 24, 2018, the treatment of loose dentition as described in commonly assigned U.S. patent application Ser. No. 16/139,444 filed on Sep. 24, 2018 and the pre-treatment of an extraction site as described in commonly assigned U.S. patent application Ser. No. 16/139,727 filed on Sep. 24, 2018.

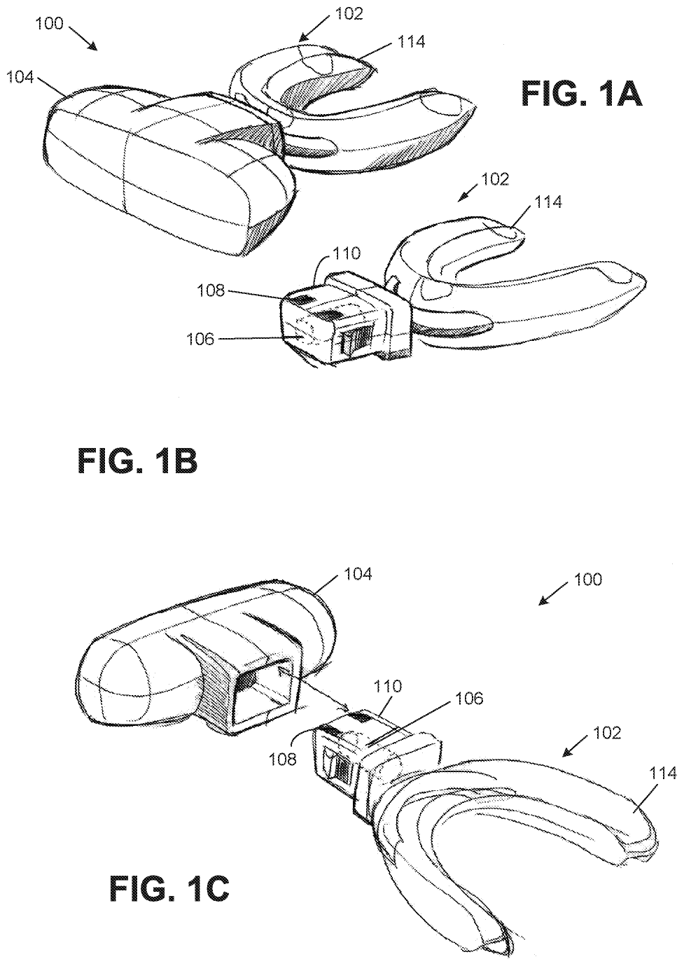

Referring to FIGS. 1A-1C, a dental device 100 includes a mouthpiece 102 having an attached base 104. The mouthpiece 102 can be separable from the base 104. The mouthpiece 102 can include a biteplate 114 (with or without a separate mouthguard thereover, as described further below) and a mouthpiece extension 110 configured to connect with the base 104. In one embodiment (as shown in FIGS. 1A-1C), the biteplate 114 can be approximately U-shaped so as to cover the occlusal surfaces of all or nearly all of the patient's teeth. Variations are contemplated, such as C-shaped or D-shaped bite plates. The mouthpiece might also be formed to contact only some of the teeth, such as shown in commonly assigned U.S. Pat. D839,435 issued on Jan. 29, 2019. Further, a motor 106 can be located in the mouthpiece 102 and configured to vibrate the mouthpiece 102. The base 104 can include the electronics necessary to run the motor 106. Contacts 108 can electrically connect the base 104 with the mouthpiece 106. In some embodiments, the vibrator can be powered by an accessory device, such as a smartphone.

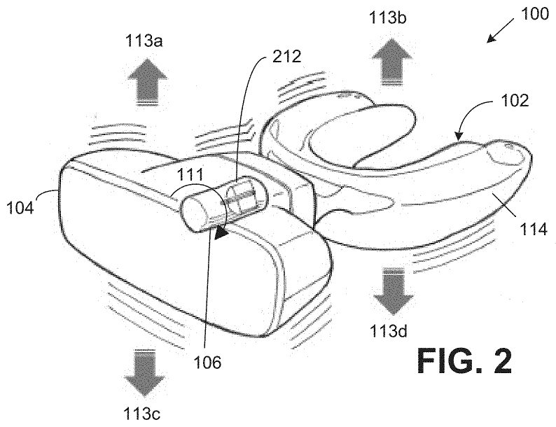

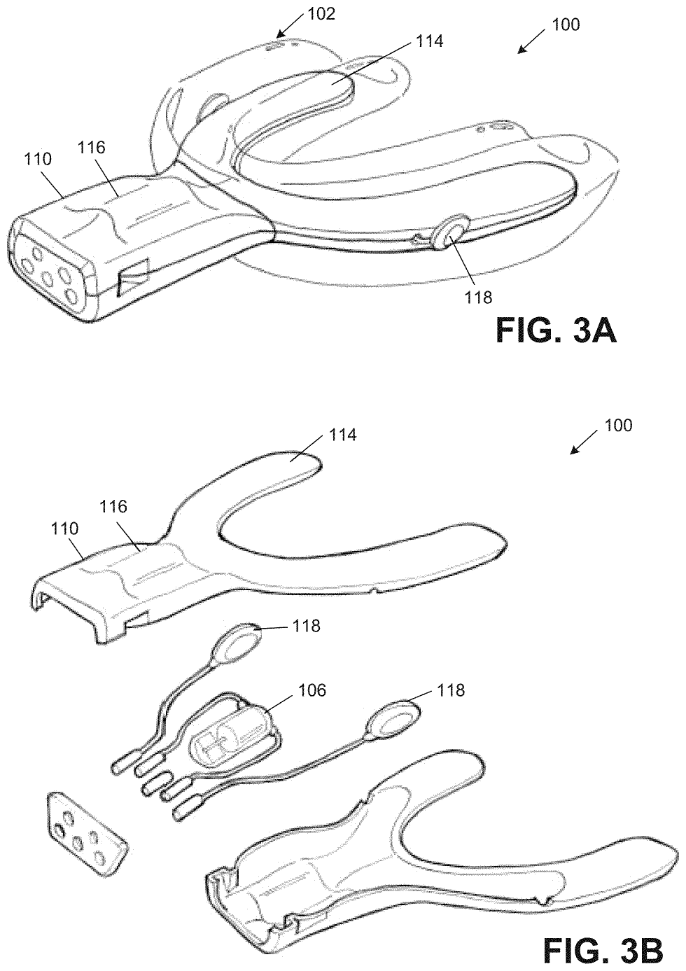

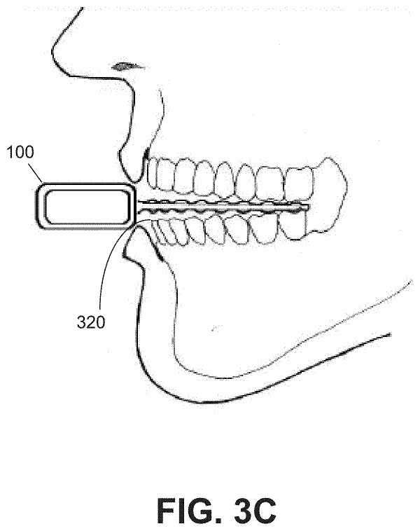

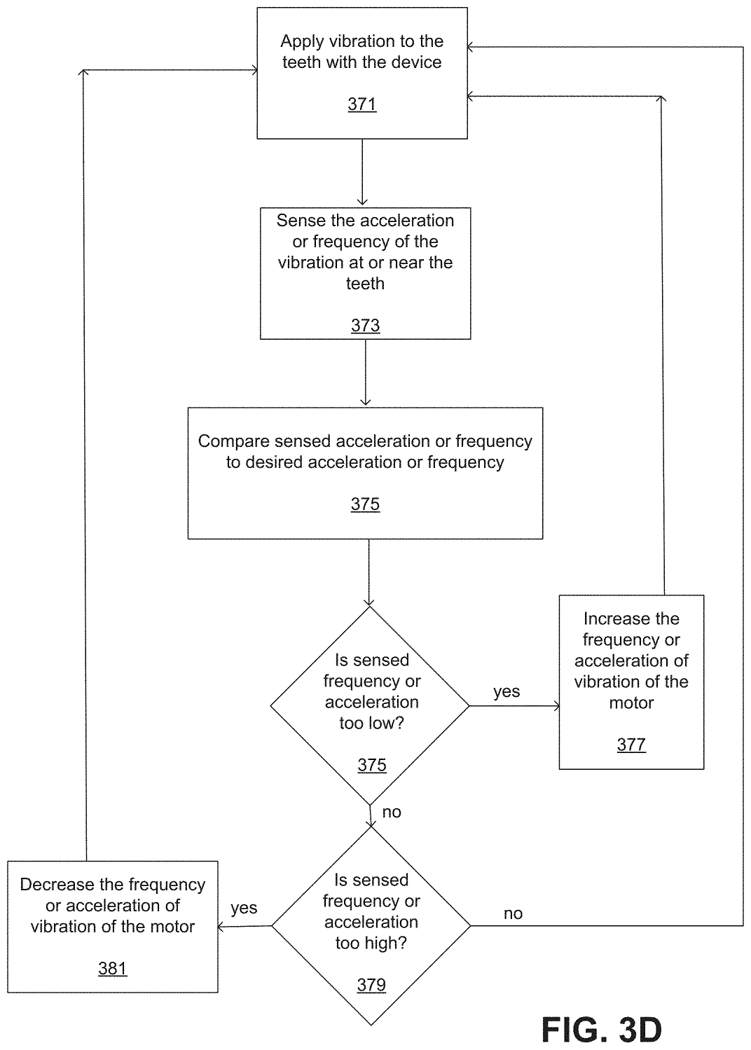

As shown in FIG. 2, the motor 106 can be a counter-weighted motor extending in-line with the extension 110 (i.e. lay horizontal with its longitudinal axis parallel to the longitudinal axis of the extension 110). The motor 106 can include a counterweight 212 that is off-axis from the longitudinal axis of the motor 106. As a result, when the motor 106 rotates, as shown by the arrow 111 in FIG. 2, the counterweight 212 moves up and down, causing the mouthpiece 102 to vibrate up and down, as shown by the arrows 113a-d in FIG. 2. Accordingly, referring to FIG. 3C, when the mouthpiece 102 is placed in a patient's mouth and the dental device is 100 turned on, the vibration of the mouthpiece 102 will place axial vibratory force on the occlusal surface 320 of the teeth, i.e., the biteplate 114 (and any guard placed thereover, as described below) will move axially away from the occlusal surface 320 of the teeth and then back onto the occlusal surface 320 of the teeth repetitively. This “smacking” up and down motion can simulate the chewing motion. By simulating the chewing motion, bone in the mouth (e.g., teeth), can be strengthened through the body's natural mechanisms, i.e., bone growth can occur due to the smacking motion.

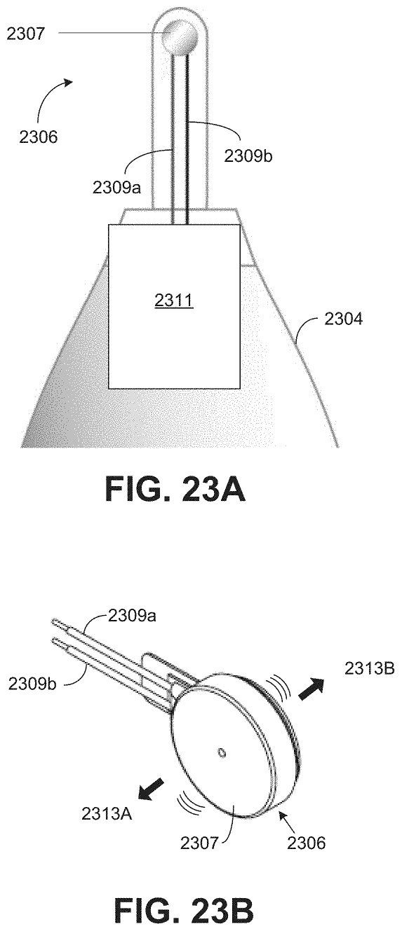

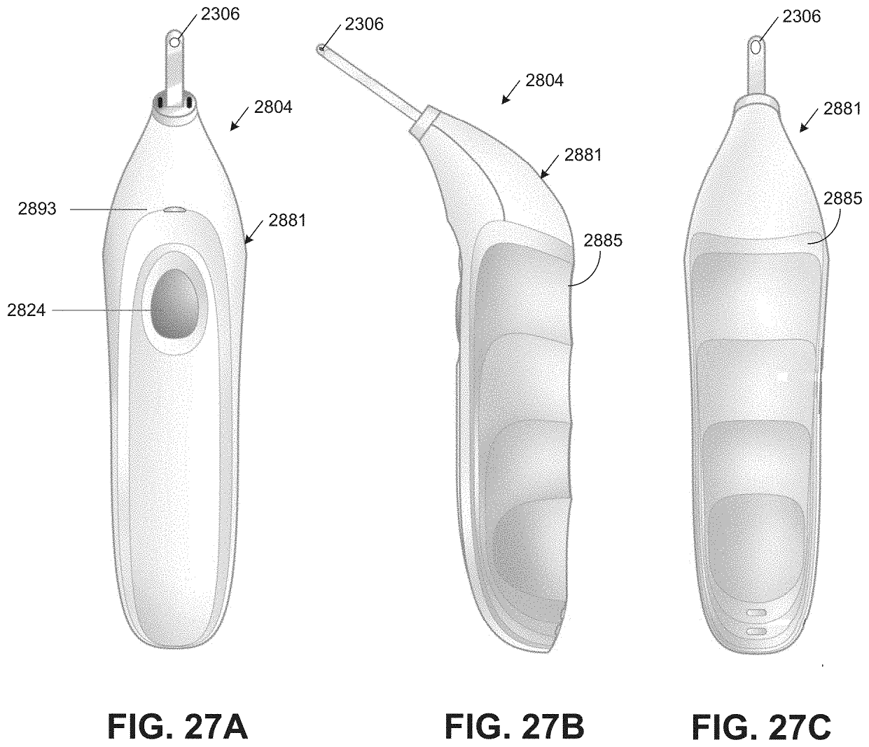

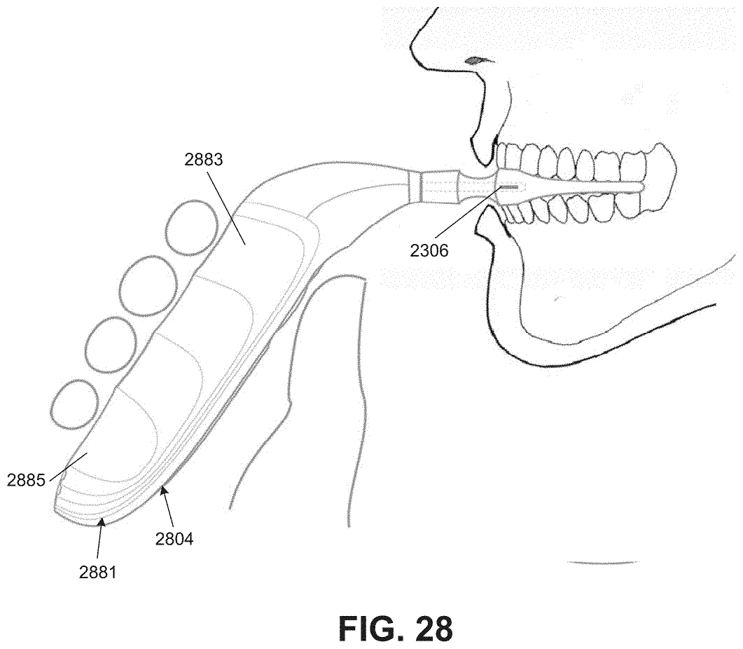

In other embodiments, as shown in FIGS. 23A-23B, the motor 106 can be replaced with a pancake motor 2306 that includes a drum 2307 that moves up and down (shown by the arrows 2313a,b in FIG. 23B). The drum 2307 can be attached to two leads 2309a,b that can connect the drum 2307 with a power source 2311. The pancake motor 2306 can be placed in an extension 2320 on the base 2304, as shown in FIG. 23A (the motor 2306 in an extension of the base is also shown in FIGS. 27A-C) or can be located with an extension on the mouthpiece. Further, in some embodiments, the pancake motor 2306 can be placed such that the motor extends just inside the teeth, as shown in FIG. 28. Similar to the motor 106, the motor 2306 can place axial vibratory force on the occlusal surface of the teeth, i.e., the mouthpiece can move axially away from the occlusal surface and then back onto the occlusal surface repetitively in a “smacking” motion.

It is to be understood that other types of motors can be used in place of motor 106 or motor 2306 to similarly cause the biteplate 114 to smack the teeth. For example, the motor could be a piezoelectric motor, a linear motor, or an electromagnetic motor. Further, it is to be understood that the motors 106 and 2306 can be interchanged for any of the embodiments described herein. The motors used for the devices described herein can advantageously be small and lightweight. For example, the motor can be less than 2 grams, such as less than 1.5 grams, such as less than or equal to 1.2 grams. Further, the motor can be configured to require low current such that the power requirements are low. For example, the voltage required for the motor to run can be less than 5 volts, such as less than 4 volts, less than 3 volts, or less than 2 volts. In some embodiments, the motor requires between 0.5 and 4 volts, such as approximately 1.5 volts. Further, the motor can advantageously consume less than 250 mW of power, such as less than 200 mW of power and/or can have an operating current of less than 100 mA, such as less than 75 mA, such as less than 65 mA. As a result, the overall device (including the mouthpiece and the base) can advantageously be less than 100 grams, such as less than 75 grams, less than 50 grams, less than 40 grams, or less than 35 grams.

The motor 106 and/or motor 2306 can be configured to vibrate the mouthpiece 102 at frequencies between 60 HZ and 130 HZ, such as between 100 HZ and 120 HZ and at accelerations of 0.035 G to 0.100 G, such as 0.050 G to 0.060 G. These frequencies and accelerations can advantageously increase bone growth in the mouth. The motors 106, 2306 can further be configured to oscillate between various vibration settings. For example, the motor 106 can oscillate between four predetermined frequencies. In one embodiment, the motor 106 oscillates between 60 hz at 0.035G, 60 hz at 0.060G, 120 hz at 0.035G, and 120 hz at 0.060G. Advantageously, by oscillating between frequency and acceleration settings, a patient's teeth will be less likely to adapt to a particular vibration setting and will continue to strengthen and grow over time.

In some embodiments, motor 106 is configured to vibrate mouthpiece 102 at a frequency higher than 80 Hz, such as at a frequency between about 60 Hz and 150 Hz, or between 100 Hz to about 140 Hz, and more specifically at a frequency at or about 120 Hz. Motor 106 may be further configured to vibrate mouthpiece 102 at an acceleration magnitude ranging between about 0.03 G and about 0.2 G. As described herein, the vibrational frequency of mouthpiece 102 may vary from the rated “free-air” vibrational frequency of motor 106 due to the amount of biting force or load applied to mouthpiece 102, such as the force used to clamp vibrational dental device 100 in place. For example, when motor 106 is configured to vibrate at a frequency of or about 120 Hz, adding biting force or load to mouthpiece 102 may result in a lower vibrational frequency of mouthpiece 102 ranging from about 100 Hz to about 120 Hz. As described herein, the frequency can oscillate, or continuously “sweep” between frequencies, or in other exemplary embodiments step between frequencies throughout the treatment period.

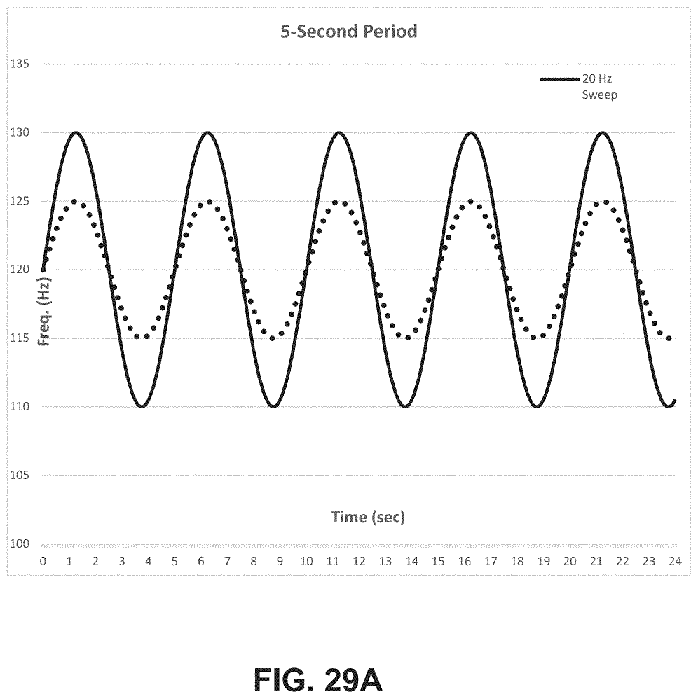

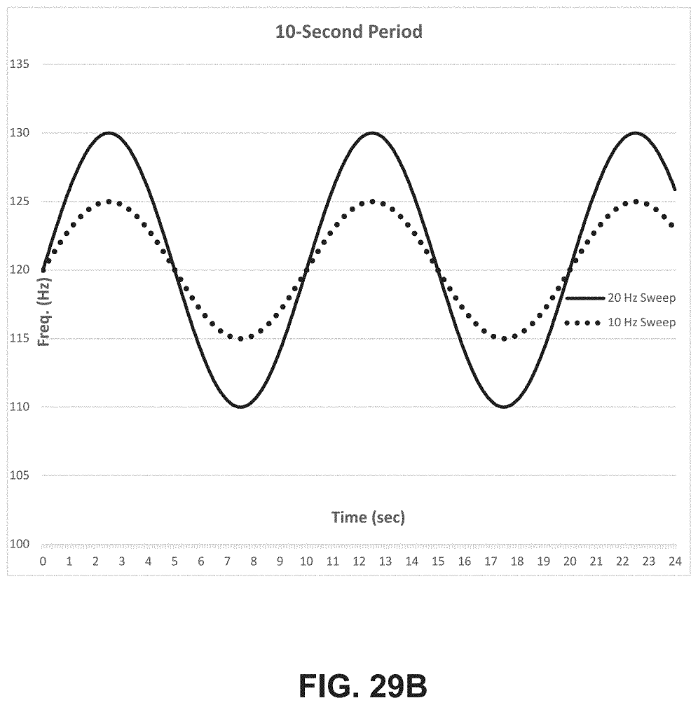

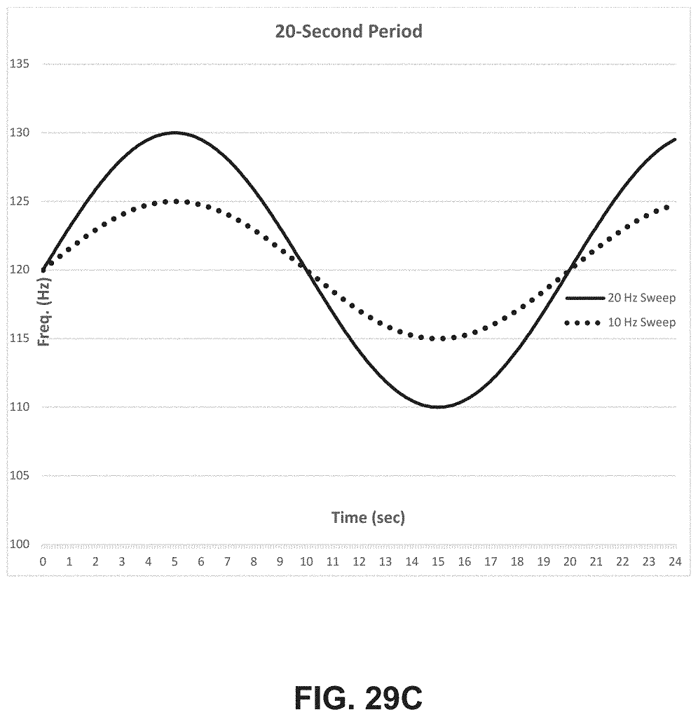

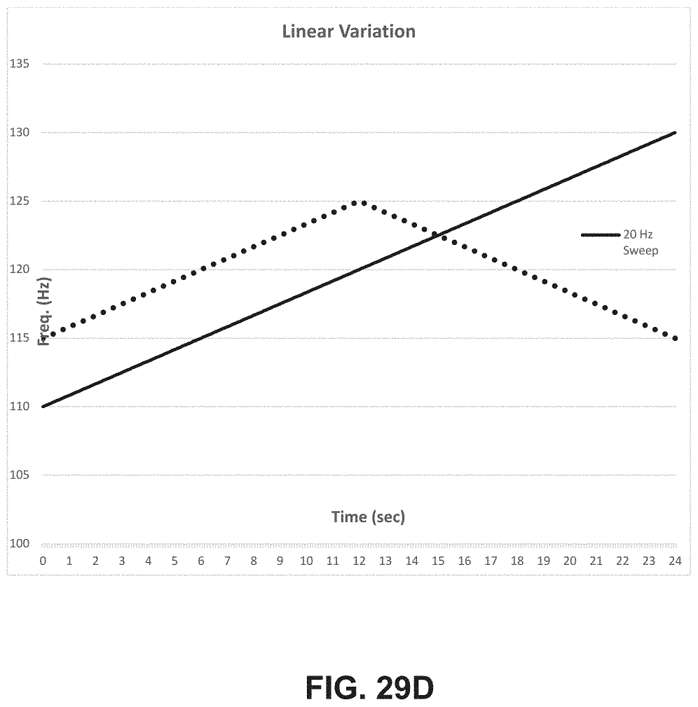

FIGS. 29A-D depict various illustrative waveforms according to the present disclosure, wherein the frequency of the vibration is substantially continuously varied. In FIGS. 29A, 29B and 29C, illustrative variations of a sinusoidally varying frequency waveform are depicted, and in FIG. 29D a linearly varying frequency waveform is depicted. It is to be understood that the continuous varying of the waveform can take any form, and that the smooth sinusoidal frequency sweep, and the linear frequency sweep, can each vary based on numerous characteristics, such as period of the frequency change, amplitude of the frequency change, center frequency, etc. Further, while the various waveforms are depicted as commencing at t=0 at the centering or base frequency, illustratively chosen to be 120 Hz, it is understood that this choice is arbitrary. In actual practice, the vibration waveform may include a ramp-up starting period, which can be of any morphology.

FIG. 29A depicts a sinusoid-shaped vibration variation pattern where the frequency varies continuously. The frequency completes a sweep between 110 Hz and 130 Hz once every 5 seconds, thus is referred to as having a period of 5 seconds. As shown, in this illustrative embodiment the frequency variation is centered about 120 Hz, increasing 10 Hz to a maximum of 130 Hz and decreasing 10 Hz to a minimum of 110 Hz. The sweep, or amplitude, is thus 20 Hz, the difference between the maximum and the minimum. Also shown in a dotted line is a plot of another illustrative frequency variation waveform where the amplitude is only 10 Hz, and the frequency cycles between 115 Hz and 125 Hz at the same period as the other waveform shown in FIG. 29A. These variations are for illustrative purposes only.

FIG. 29B depicts another sinusoid-shaped vibration variation pattern where the frequency varies continuously. The frequency completes a sweep between 110 Hz and 130 Hz once every 10 seconds, thus is referred to as having a period of 10 seconds. As shown, in this illustrative embodiment the frequency variation is centered about 120 Hz, increasing 10 Hz to a maximum of 130 Hz and decreasing 10 Hz to a minimum of 110 Hz. The sweep, or amplitude, is thus 20 Hz, the difference between the maximum and the minimum. Also shown in a dotted line is a plot of another illustrative frequency variation waveform where the amplitude is only 10 Hz, and the frequency cycles between 115 Hz and 125 Hz at the same period as the other waveform shown in FIG. 29B. These variations are for illustrative purposes only.

FIG. 29C depicts another sinusoid-shaped vibration variation pattern where the frequency varies continuously. The frequency completes a sweep between 110 Hz and 130 Hz once every 20 seconds, thus is referred to as having a period of 20 seconds. As shown, in this illustrative embodiment the frequency variation is centered about 120 Hz, increasing 10 Hz to a maximum of 130 Hz and decreasing 10 Hz to a minimum of 110 Hz. The sweep, or amplitude, is thus 20 Hz, the difference between the maximum and the minimum. Also shown in a dotted line is a plot of another illustrative frequency variation waveform where the amplitude is only 10 Hz, and the frequency cycles between 115 Hz and 125 Hz at the same period as the other waveform shown in FIG. 29C. These variations are for illustrative purposes only.

FIG. 29D depicts another vibration variation pattern where the frequency varies continuously, only linearly. The frequency completes a linear sweep between 110 Hz and 130 Hz in 24 seconds. As shown, in this illustrative embodiment the frequency variation is centered about 120 Hz, increasing 20 Hz from 110 Hz to a maximum of 130 Hz. The sweep is thus 20 Hz, the difference between the maximum and the minimum. Also shown in a dotted line is a plot of another illustrative frequency variation waveform where the sweep is only 10 Hz, and the frequency cycles between 115 Hz and 125 Hz in 12 seconds, one-half the time period as the other waveform shown in FIG. 29D. These variations are for illustrative purposes only.

In some embodiments, as shown in FIGS. 3A-3B, the device 100 can include sensors 118, such as piezoelectric sensors, configured to detect the acceleration or frequency of the vibration just proximate to the occlusal surfaces of the teeth. The sensors 118 can be placed, for example, on the outside or the inside of the biteplate. The sensors 108 can be connected to circuitry that includes a feedback loop for running the motor 106. That is, when the mouthpiece 102 touches the teeth, the surface contact and/or force between the mouthpiece 102 and the teeth can dampen the vibrations and/or slow the motor down. The feedback loop can therefore be used to compensate for the slowed motor.

Referring to FIG. 3D, a feedback loop can thus include applying vibration to the teeth with a dental device (such as device 100 or any device described herein) at step 371. The acceleration or frequency of the vibration can be sensed or measured at step 373 at or near the teeth, such as with the sensors 118. The sensed acceleration or frequency can be compared to the desired acceleration or frequency at step 375. At step 375, it can be determined whether the frequency or acceleration is too low. If so, then the frequency or acceleration can be increased at step 377. If not, then it can be determined whether the sensed frequency or acceleration is too high at step 379. If so, then the frequency or acceleration can be decreased at step 381. The feedback loop can then repeat. Thus, the acceleration or frequency of the vibration at the motor can be adjusted to obtain the desired acceleration or frequencies at the mouthpiece 102 regardless of the dampening effect caused by interaction with the teeth.

In one embodiment, shown in FIGS. 3A-3B, the motor 106 can be located within the extension 110 of the mouthpiece 102. Thus, for example, the extension 110 can have a pocket 116 to house the motor 106. The motor 106 can be placed close to the biteplate 114, such as within 1 mm of the biteplate 114, so that the motor 106 is located at least partially within the patient's mouth, i.e., is located intraorally (see FIG. 3C). For example, the counterweight 212 causing the vibration can be positioned so as to be located within the patient's mouth when the dental device 100 is in use. Having the motor 106 located intraorally advantageously both increases the ability of the mouthpiece 212 to smack against the occlusal surfaces of the patient's teeth and avoids having the device extend too far outside of the mouth, which can cause discomfort to the patient if the base is intended to be used without hands.

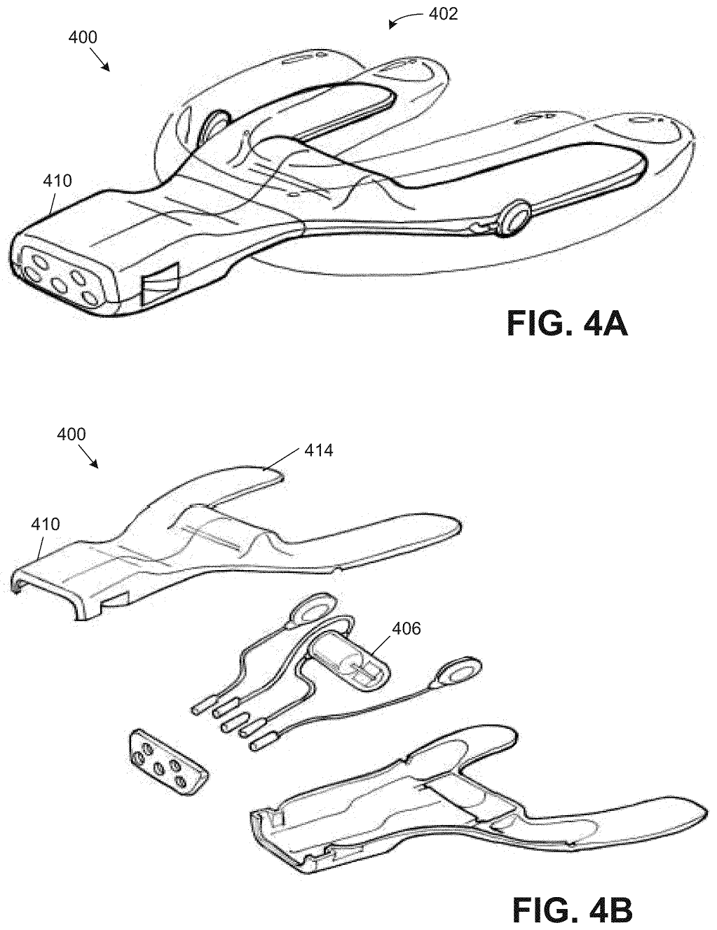

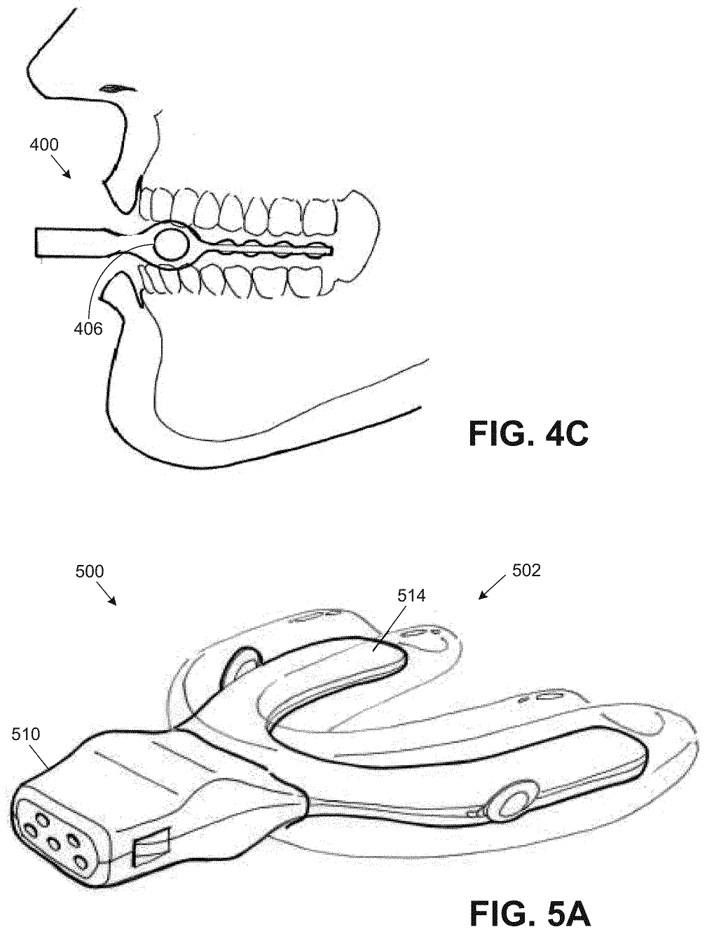

Although the motor has been described as inside of and inline with the extension 410 of the mouthpiece 102, other configurations are possible. For example, referring to FIGS. 4A-4B, in one embodiment, a dental device 400 can have a motor 406 that is located inside of the biteplate 414. Further, the motor 406 can lay horizontal within the extension 410, but be placed such that its longitudinal axis extends perpendicular to the long-axis of the extension 410. The horizontal configuration of the motor still allows the counterweight 212 to provide a smacking motion while the perpendicular configuration allows the motor 406 to be located inside the teeth of a patient's mouth, for example sitting up against the roof of the mouth.

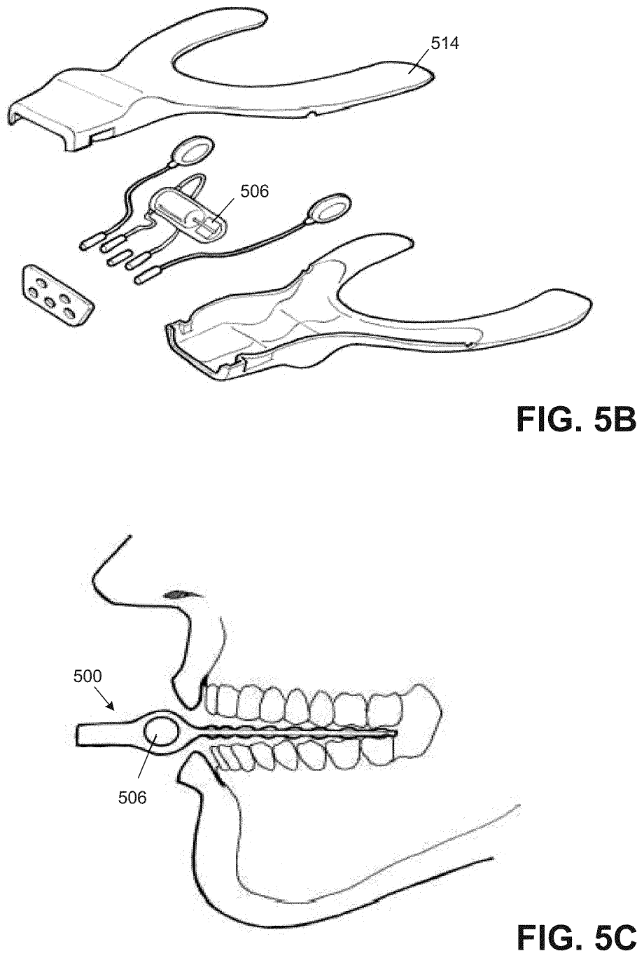

Likewise, referring to FIGS. 5A-5B, the dental device 500 can have a motor 506 that is located inside of the extension and that lays horizontal and perpendicular to extension 510. As described above, the horizontal configuration of the motor allows the counterweight 212 to provide a smacking motion, thereby enhancing tooth growth.

In some embodiments, the motors described herein can include an insulator therearound, such as a ceramic sleeve.

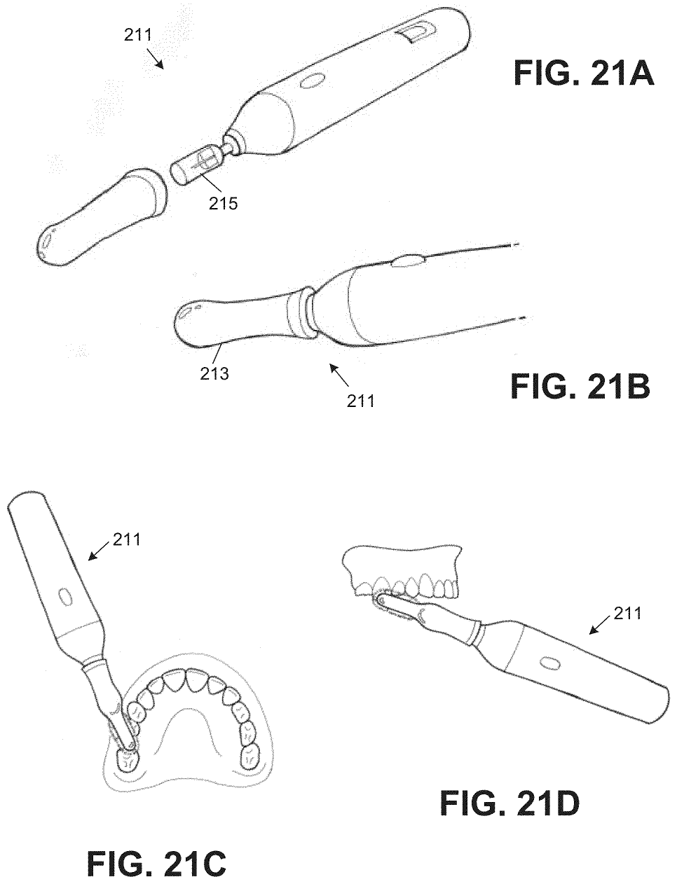

Referring to FIGS. 21A-21D and 24A-26C, the devices described herein need not include a mouthpiece configured to cover all of the teeth. Rather, mouthpieces specifically targeting particular teeth can be used. It is to be understood that the mouthpieces shown and described with respect to FIGS. 21A-21D and 24A-26C can be used with any of the motors, bases, and guards described herein.

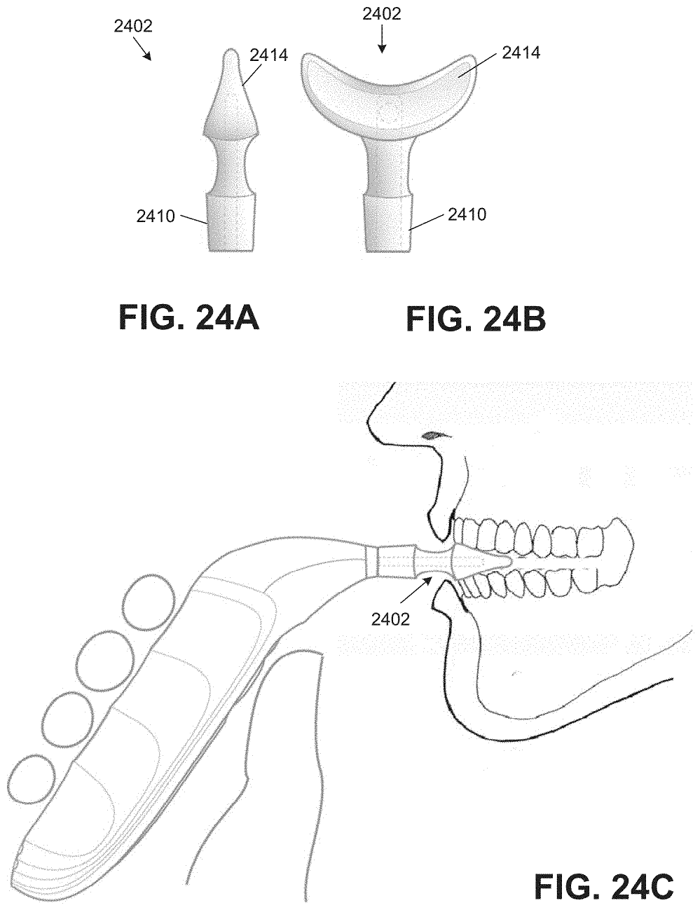

For example, referring to FIGS. 24A-C, a mouthpiece 2402 can have a crescent-shaped biteplate 2414 configured to cover the social six teeth. Such a design can be advantageous, for example, for treating crowding in the social six teeth.

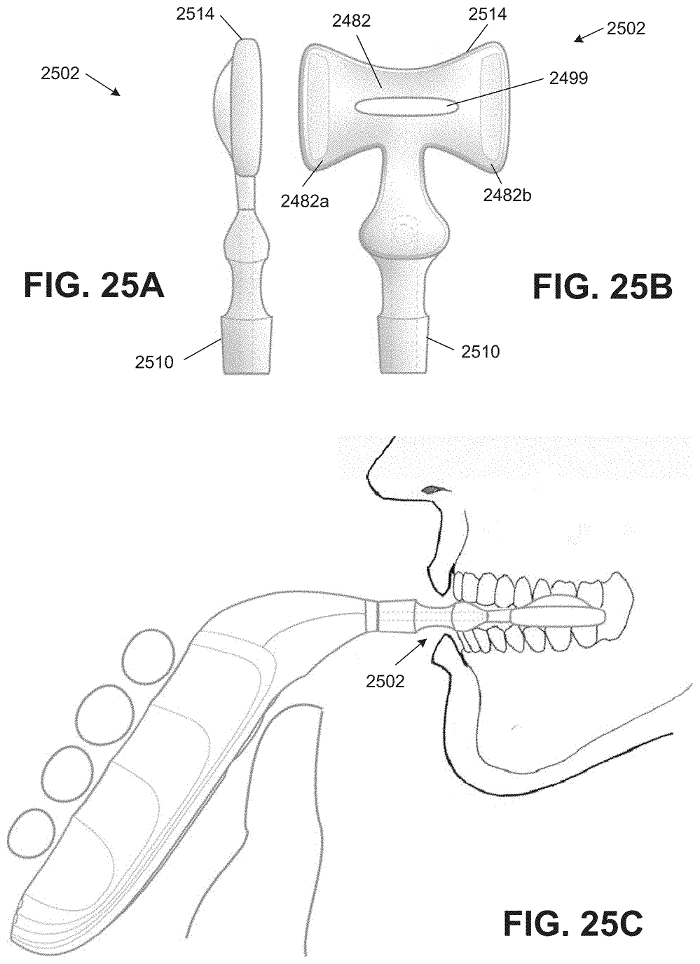

As another example, referring to FIGS. 25A-25C, a mouthpiece 2502 can have a double-hammer-shaped biteplate 2514 configured to cover only the molars. Such a design can be advantageous, for example, for treating molar protraction or retraction. The biteplate 2514 can thus include a narrow central portion 2482 configured to rest on the tongue and two elongated edge portions 2484a,b configured to rest on the occlusal surfaces of the molars. Further, the central portion 2482 can include a convex section 2499 configured to sit over the lounge for comfort and ease of use.

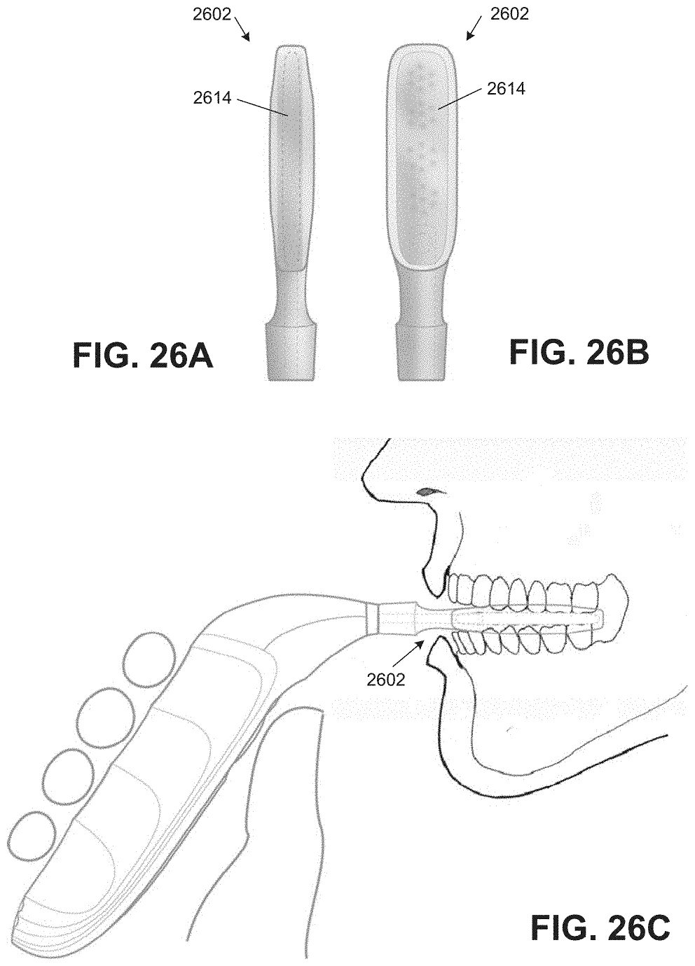

As another example, referring to FIGS. 26A-26C, a mouthpiece 2602 can have an elongate biteplate 2614. The elongate biteplate 2614 can be configured to be placed on one side of the mouth and/or one quadrant of the teeth.

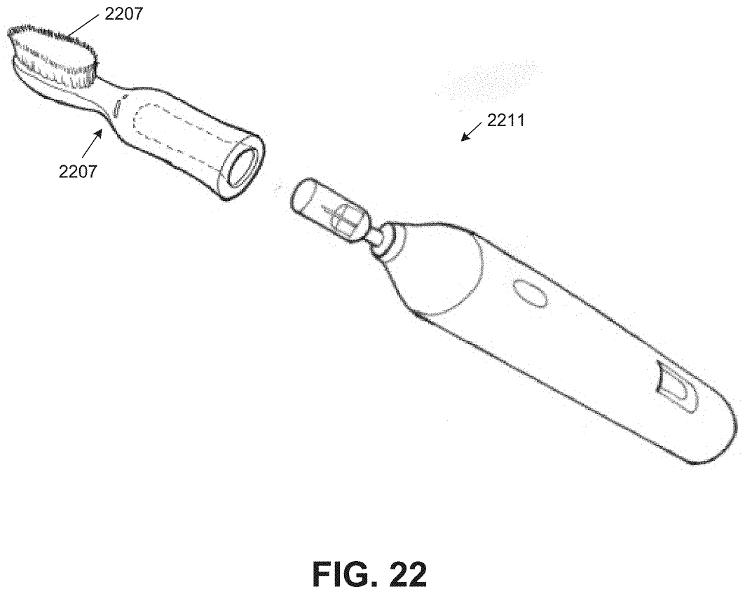

As another example, in one embodiment, shown in FIGS. 21A-21D, the device 211 can include a rounded end or nub 213. The nub 213 can include the motor 215 therein, which can be configured similarly to the motors described above. As shown in FIG. 21C-21D, by having only a nub 213 rather than a full mouthpiece, specific individual teeth in need of treatment can be targeted. Variations on the nub are possible. For example, referring to FIG. 22, the nub 2213 on device 2211 can include a brush 2207 on the end configured to provide a more gentle vibratory force on the teeth.

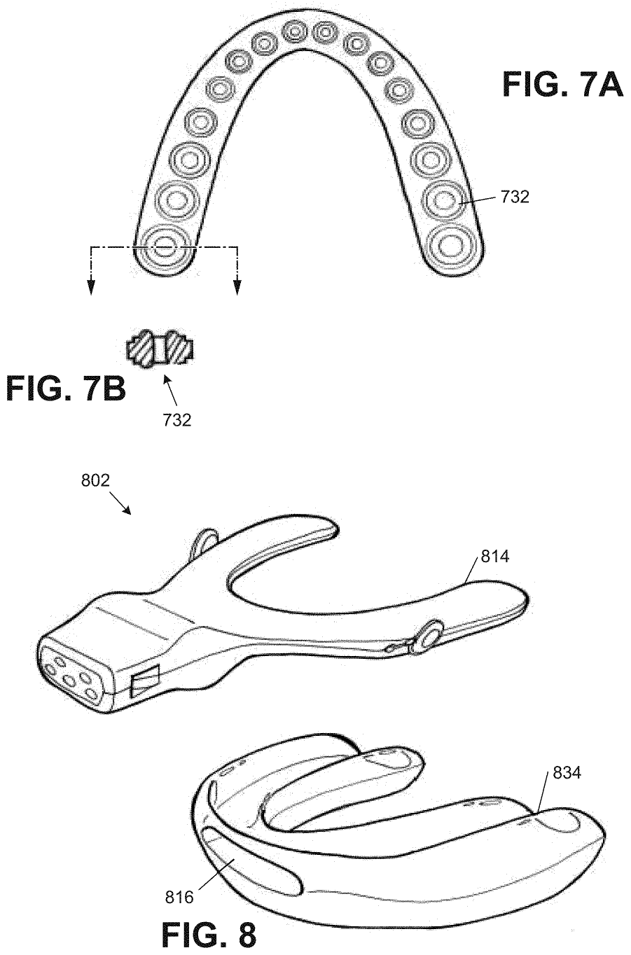

Referring to FIGS. 7A and 7B, the biteplate 714 for any of the mouthpieces described herein can include raised dimples 732, or outward extensions. There can be approximately one dimple 732 for each tooth intended to be treated. Further, the dimples 732 can be spaced apart in such a manner as to approximately align with the center of some or all of the occlusal surfaces of a patient's teeth when the mouthpiece is in use. The dimples 732 can advantageously help the mouthpiece effectively smack the teeth by providing an extended point of contact to ensure that contact is made with each tooth. In some embodiments, the dimples 732 can be customized to a patient's tooth alignment. Each dimple 732 can have a peak that has a surface area of less than 70%, such as less than 50%, of the surface area of the corresponding tooth so as to place pressure on less than 75% or less than 50% of each tooth.

Referring to FIG. 8, the mouthpiece 802 (which can correspond to any mouthpiece described herein) can include two separable parts, the biteplate 814 and a mouthguard 834. The biteplate 814 can be made of a hard material, such as metal. The mouthguard can be made of a softer material such as a polymer.

In some embodiments, the mouthguard 834 can be custom fit to the patient's mouth. By having a custom fit mouthguard 834, the mouthpiece 802 can be more efficient and effective in applying the vibratory smacking force on a patient's teeth. As shown in FIG. 8, the mouthguard 834 can include a hole 836 which can be used to place the mouthguard 834 over the biteplate 814 after formation.

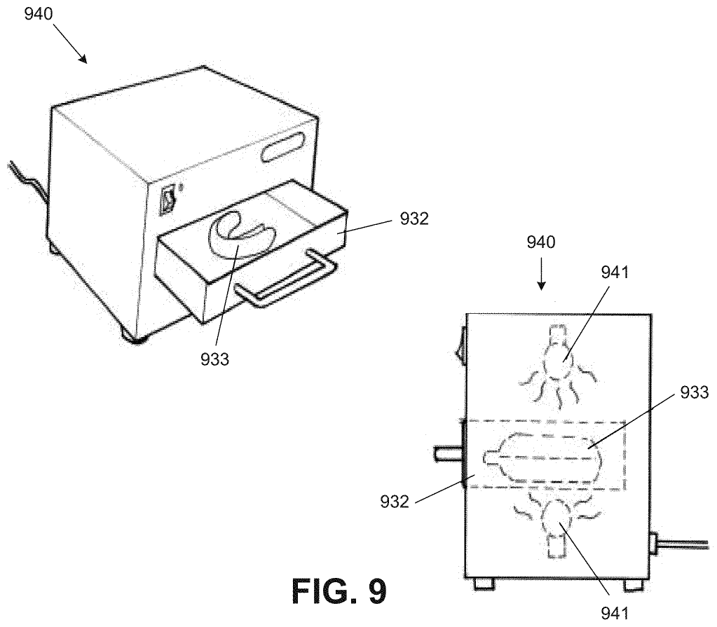

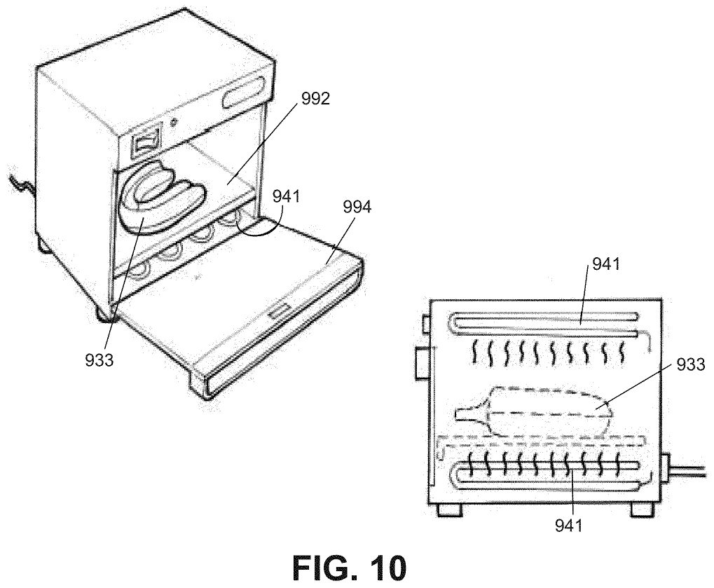

Referring to FIG. 9, the mouthguard 834 can be produced quickly and easily on-site, e.g., at a dentist's office, within minutes by using an oven 940. To form a mouthguard 834 using the oven 940, the mouthguard 834 can be made of a material such as silicone or an ethylene vinyl acetate copolymer, e.g., Elvax®, that is easily formable once warm. The oven 940 can include a heat source 941, such as infrared bulbs, a heat lamp, or heating coils, configured to heat up the mouthguard 814. A mouthguard preform 933 (i.e. one not yet formed to the teeth) can be placed around a biteplate (which can be any of the biteplates described herein) and in the oven 940. The mouthguard preform 933 and biteplate can be exposed to the heat source 941 for between 1 and 10 minutes at temperatures of between 120° and 200° F., less than 200°, or less than 175°. Advantageously, as the mouthguard preform 933 warms, it can become slightly softer, thereby conforming to the shape of any dimples in the biteplate without losing its overall shape. Further, once the mouthguard preform 933 is warmed up sufficiently, the user can take the mouthguard preform 933 out of the oven 940 and have the patient bite down, leaving an impression of the teeth in the mouthguard preform 933. Advantageously, by using temperatures of between 120° and 200° F., less than 200°, or less than 175° to heat the mouth guard, the mouthguard preform 933 will be cool enough upon entering a patient's mouth to not burn the patient (in contrast to temperatures, for example, of over 212°). After the patient has bit down, and as the mouthguard preform 833 cools, it will retain its shape, thereby forming the final mouthguard 834.

The oven 940 can have a variety of configurations. In some embodiments, the oven 940 is relatively small such that it can easily sit on a counter or table at the office. In some embodiments, the oven 940 can include a drawer 932 with a handle, and the drawer 932 can be configured to hold the mouthguard preform 933. In another embodiment, the oven 940 can include a shelf 992 and a hinged door 994. The oven 940 can further include a power switch, an indicator light, a timer, and/or a display to enhance ease of use.

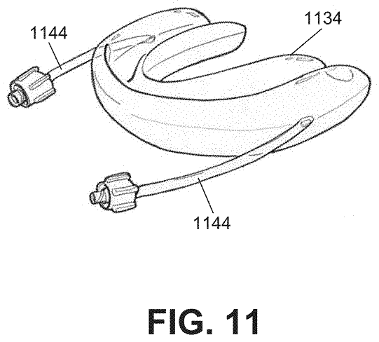

In some embodiments, shown in FIG. 11, the mouthguard 1134 can have vacuum ports 1144 to provide suction to exactly fit the mouthguard 1134 to all of the surfaces of the teeth before the mouthpiece 1134 cools completely. The vacuum ports 1144 can be removed after the mouthguard 1134 is fully formed.

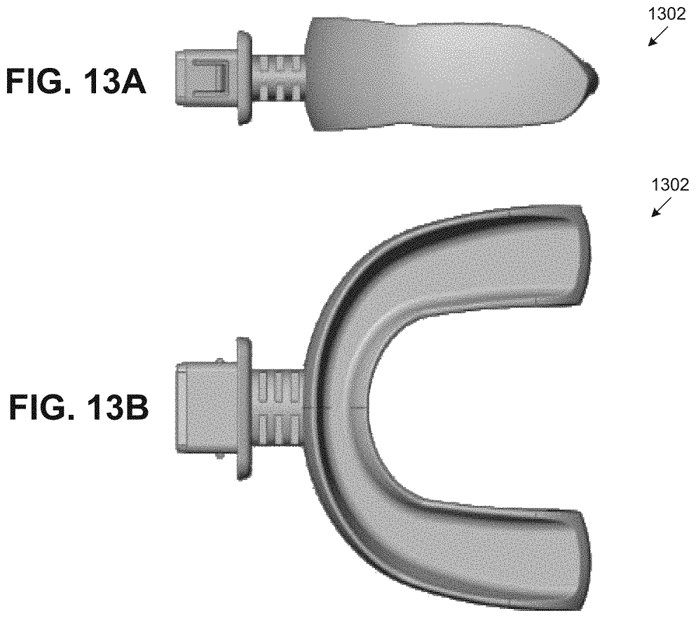

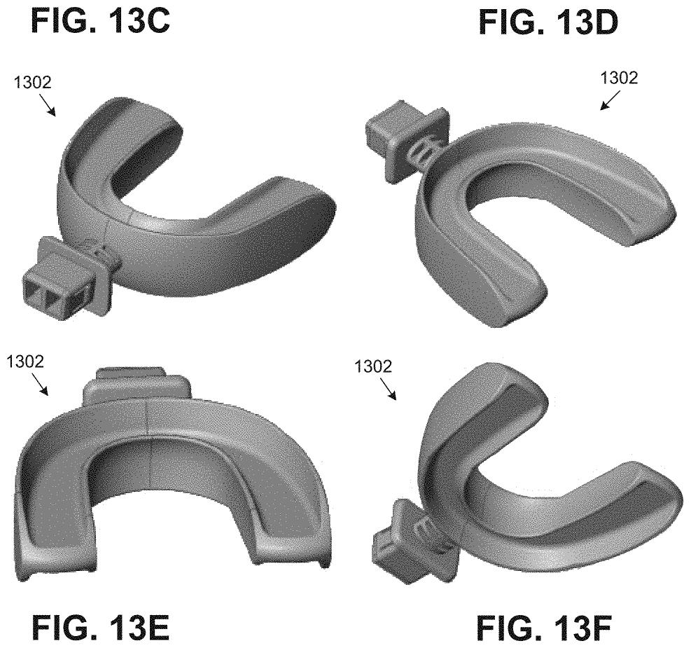

As shown in FIGS. 13A-13F, a mouthpiece 1302 of the dental devices described herein need not be formed to a patient's mouth, but can have a set shape. Further, as shown in FIGS. 13A-13F, the mouthpiece need not include a separate biteplate and mouthguard. Rather, the mouthpiece can be formed of a single piece.

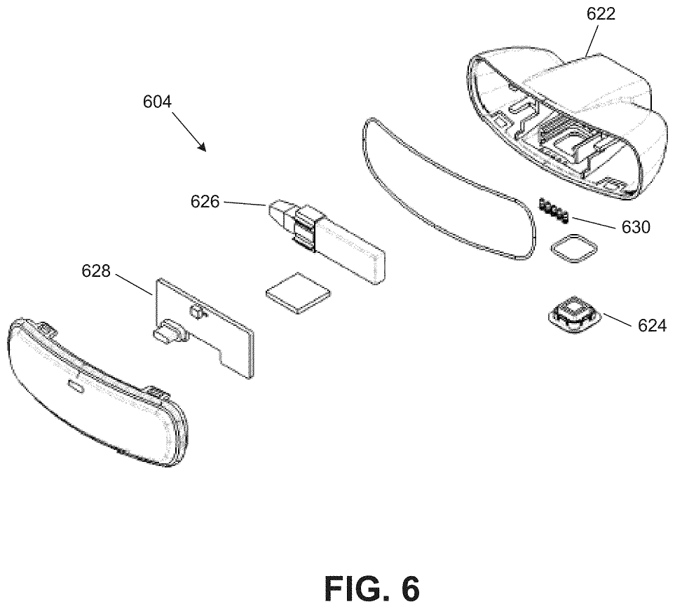

Any of the mouthpieces described herein can be connected to a base, such as base 104 or an alternative base. For example, referring to FIG. 6, a base 604 can be connected to any of the mouthpieces described herein. The base 604 can include a housing 622, an on-off switch 624 to control the vibration, electrical contacts 630 to electrically connect the base 604 with a mouthpiece, a battery 626 to power the motor, and a circuit board 628 to control the motor. The base 604 can be shaped such that it is easily held by a patient's hand. In one embodiment, the base 604 is small and light enough that it does not need to be gripped by the patient during use of the device.

As another example, referring to FIGS. 27A-28, a base 2804 can be connected to any of the mouthpieces described herein. The base 2804 can include a handle 2881 configured to be easily held by a single hand and a mouthpiece connector 2887. The handle 2881 can include a grip portion 2885 that can include indents 2883, such as four indents, configured to provide comfortable resting spot for a person's fingers when gripping the handle 2881. As shown in FIG. 28, the handle 2881 can be curved such that the grip portion 2885 can be gripped with a hand without having to tilt the device forward or up. For example, the angle between the grip portion 2885 and the mouthpiece connector 2887 can be between 30 and 60 degrees, such as approximately 45 degrees. Referring back to FIGS. 27A-27C, the base 2804 can house the power source, such as a battery, for the motor therein. The base 2804 can include an on-off switch 2824 to control the vibration. Further, in some embodiments, the base 2804 can include a battery indicator light 2893 thereon to indicate the amount of battery left. In some embodiments, the base 2804 can also include contacts 2891 thereon to interact with a charging station, as described below.

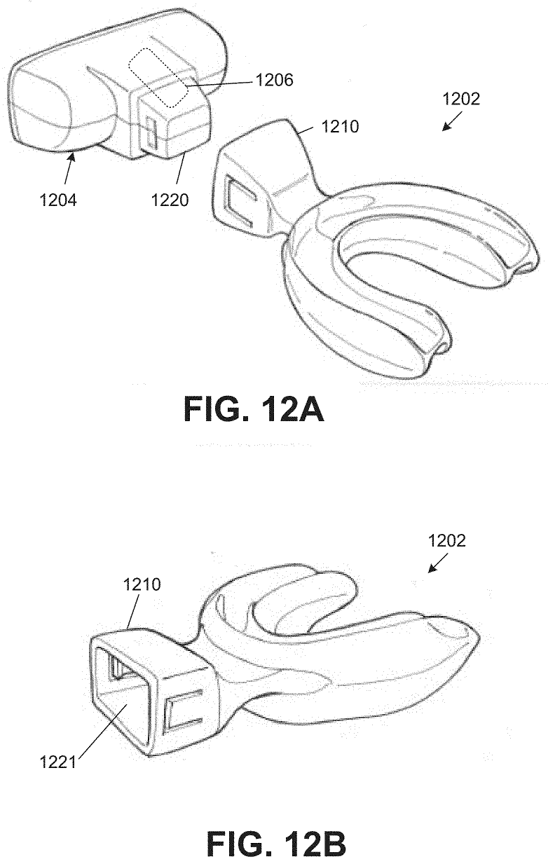

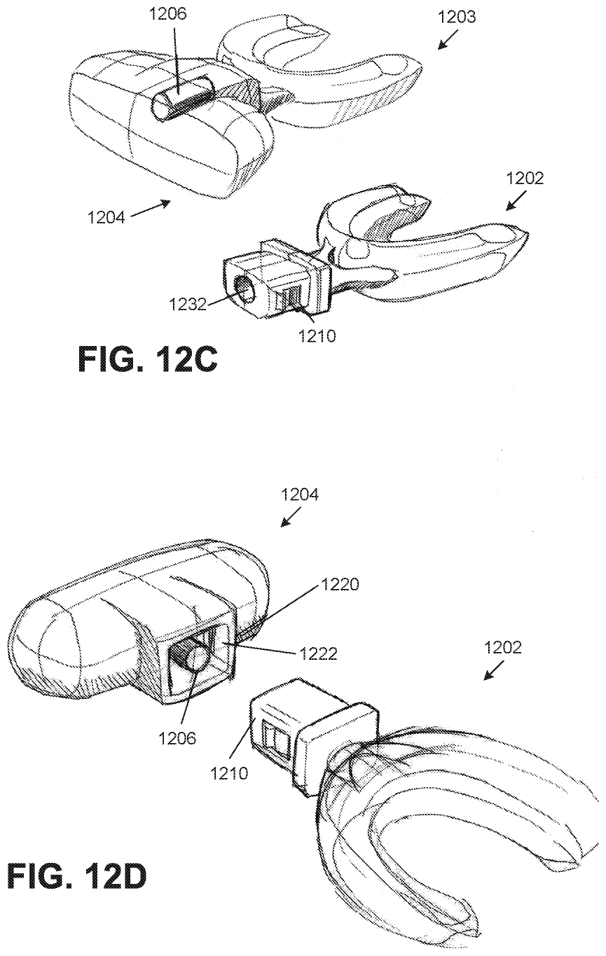

Referring to FIGS. 12A-12D, another exemplary base 1204 can be used with any of the mouthpieces described herein. As shown in FIGS. 12A-12D, the base 1204 can include a motor 1206 therein (in place of or in addition to the motor in the mouthpiece). By including the motor in the base, there is advantageously more room for the connection to the battery while allowing the mouthpiece to be as slim as possible. For example, the mouthpiece 1202 can be free of a motor.

As shown in FIGS. 12A-12D, and 18-20 the mouthpieces can be configured to connect to the base in a variety of ways. For example, as shown in FIGS. 12A-12B, the base 1204 can include an extension 1220 to house the motor 1206, while the extension 1210 of the mouthpiece 1202 can include a hole 1221 therein to fit over or house the extension 1220 of the base 1204. In contrast, in reference to FIGS. 12C-12D, the base 1204 can include an extension 1220 having a hole 1222 therein that both holds the motor 1206 and engages with our houses the extension 1210 of the mouthpiece 1202. The extension 1210 of the mouthpiece 1202 can include a corresponding cut-out 1232 to fit over the motor 1206 when it is snapped into the base 1204.

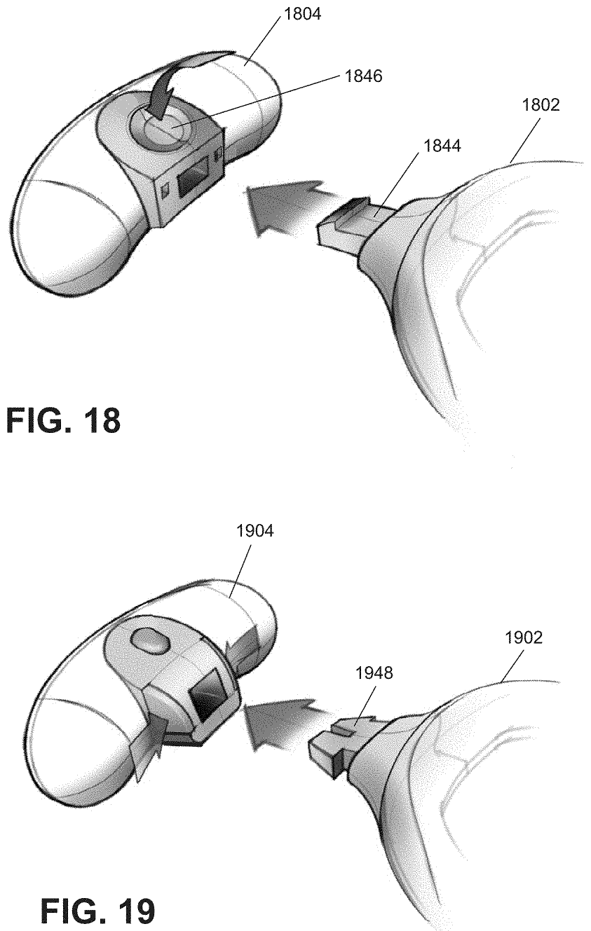

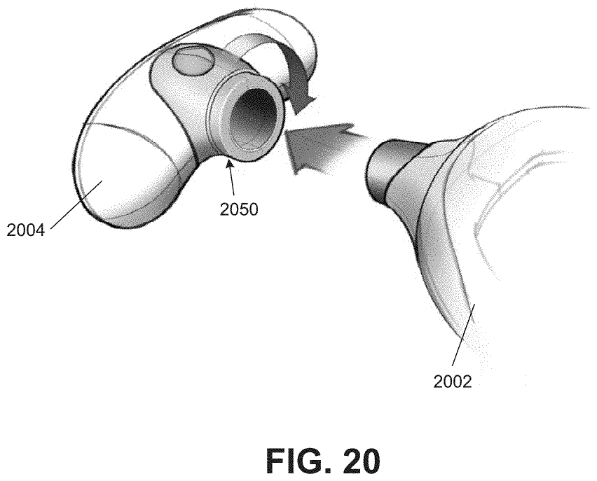

In one embodiment, as shown in FIG. 18, the base 1804 and the mouthpiece 1802 can be attached together with a mechanical connector 1844 that can set the orientation of connection and that can be released through a release button 1846. In another embodiment, shown in FIG. 19, the base 1904 and the mouthpiece 1902 can be attached together through a fork-type mechanical connection 1948; squeezing the fork portions together can lock or unlock the connection 1948. In yet another embodiment, shown in FIG. 20, a tightening collar 2050 can be used to connect a base 2004 and mouthpiece 2002.

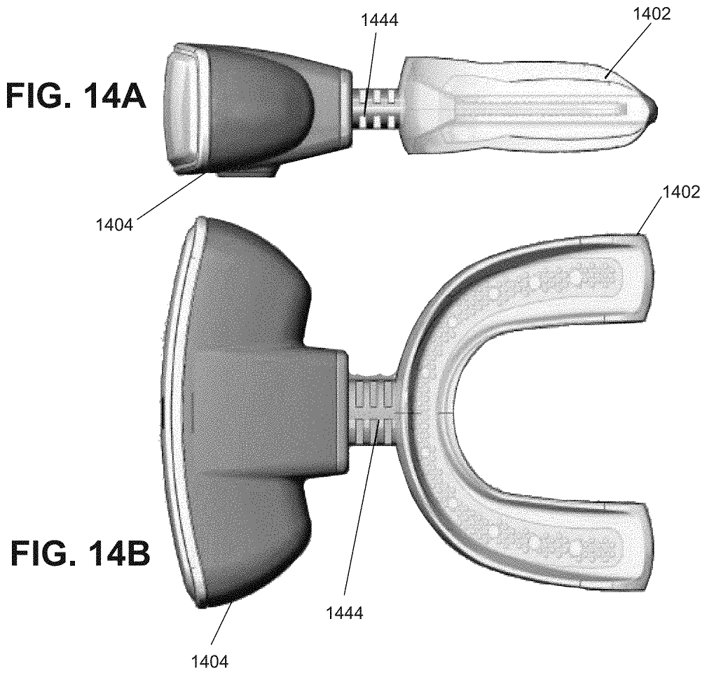

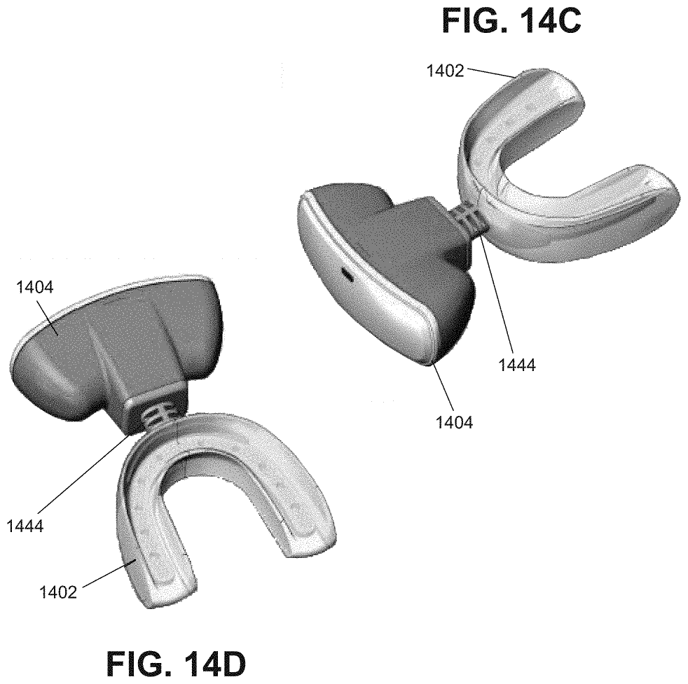

Further, as shown in FIGS. 14A-14B, in some embodiments, the dental devices described herein can include a flexible portion 1444 between the mouthpiece 1402 and the base 1404. For example, the flexible portion 1444 can include a series of cut-outs that allow the portion 1444 to easily bend. The flexible portion 1444 to provide enhanced comfort to the patient, for example, by limiting the amount of vibration that occurs outside of the mouth and by reducing the amount of torque that occurs on the mouth through the bite plate if the base is torqued suddenly. The flexible portion can have an oval-like cross-section that easily conforms to the patient's mouth, thereby enhancing the comfort of the patient.

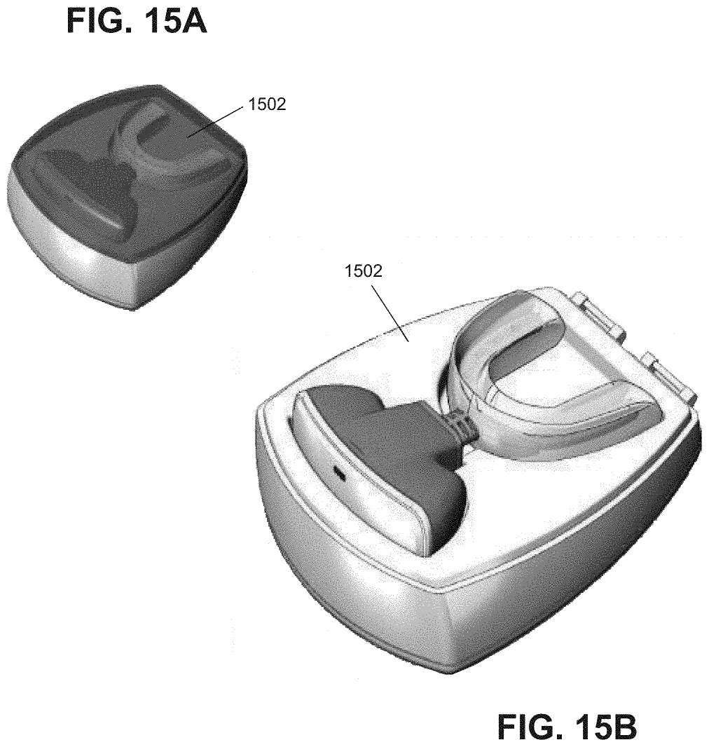

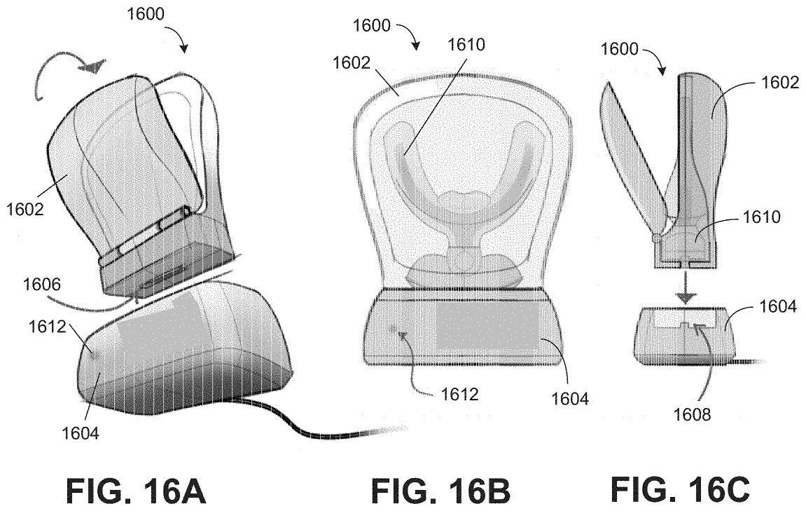

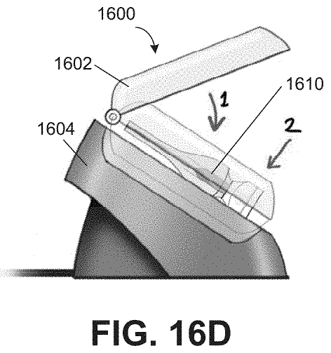

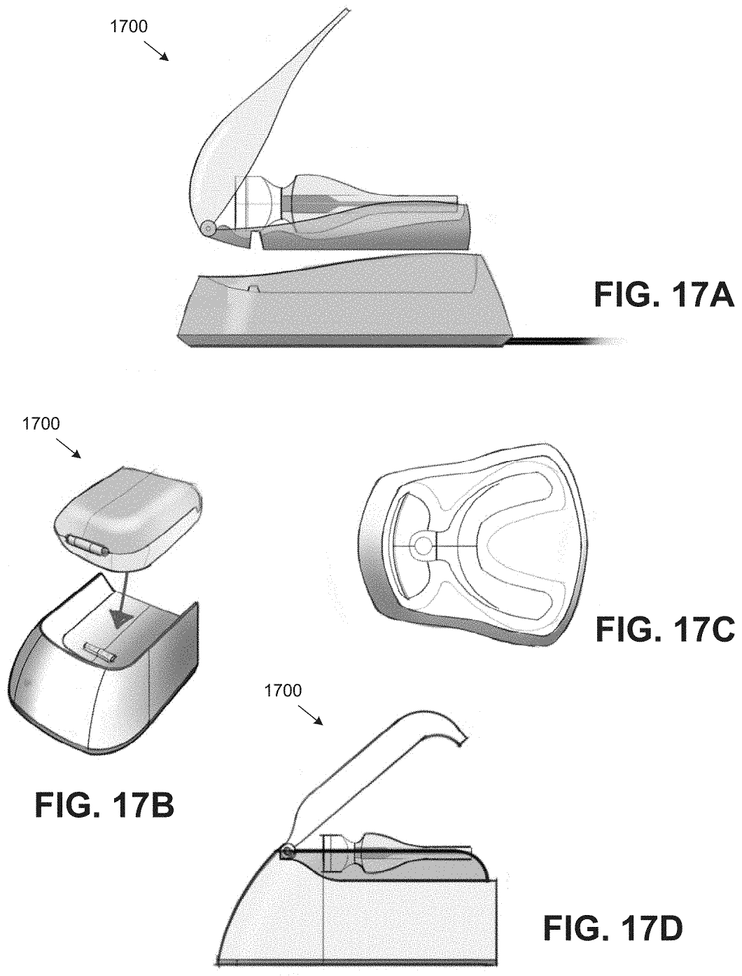

As shown in FIGS. 15-17, the devices described herein can be configured to be charged in a charging station, for example using a standard mini usb connection. As shown in FIG. 15A, the charging station can include a protective covering 1502 configured to protect the device while not in use. The protective covering 1502 can then be placed in a charging base (not shown in FIGS. 15A-15B). As shown in FIGS. 16A-16D, the charging station 1600 can include a protective covering 1602 and a charging base 1604. A connector slot 1606 can be used to sit the case 1602 in the charging base 1604. As shown in FIG. 16C, charging pins 1608 can connect from the charging base 1604 through the protecting covering 1602 and into the device 1610 to charge the device. An indicator light 1612 can indicate whether the charging station 1600 is charging. A similar station 1700 is shown in FIGS. 17A-17D. It is to be understood that other sizes, shapes, and types of charging stations could be used.

Once formed and assembled, the dental devices described herein can be used to strengthen the bone around teeth and tighten the ligaments around teeth such as for retention, e.g. orthodontic retention after braces are removed. For example, the device can be placed in the mouth for less than 10 minutes per day, such as less than 6 minutes, such as approximately 5 minutes, less than 5 minutes, or less than 1 minute per day for less than or equal to 180 days, less than or equal to 120 days, or less than or equal to 90 days to tighten the periodontal ligament after orthodontics. Such use can be in addition to or in place of traditional retainers. Use of the device can advantageously significantly decrease the time required for tightening of the periodontal ligament (from the average of six months to a year). Further, in some embodiments, the dental device can also be used for less than 2 minutes per day, such as less than 1 minute per day, on a continuing basis to provide general tooth strengthening. Further, the dental devices described herein can also be used for strengthening bone during dental implant procedures, tightening ligaments, strengthening bone after periodontics cleaning and procedures, such as after bone grafting.

Variations on the devices described herein are possible. For example, in some embodiments, the devices can have a microchip or Bluetooth connected thereto to record when and how long the device was used for. Further, it is to be understood that the various elements of the mouthpieces and bases described herein with reference to specific embodiments could be substitute and/or combined with other embodiments described herein.

Additional details pertinent to the present invention, including materials and manufacturing techniques, may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are a plurality of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware and software, but systems and methods consistent with the present disclosure can be implemented as hardware alone. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps and/or inserting or deleting steps.

Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A dental device comprising:
a mouthpiece configured to contact occlusal surfaces of a patient's teeth; and
a vibrator connected to the mouthpiece,
a controller configured to adjust the vibrator to vibrate the mouthpiece at a frequency that varies substantially continuously.

2. The dental device of claim 1, wherein the frequency varies about a base frequency of 120 Hz.

3. The dental device of claim 1, wherein the frequency varies between 110 Hz and 130 Hz.

4. The dental device of claim 1, wherein the frequency varies between 115 Hz and 125 Hz.

5. The dental device of claim 1, wherein the frequency varies sinusoidally.

6. The dental device of claim 1, wherein the frequency varies linearly.

7. The dental device of claim 1, wherein the acceleration is between 0.03 G and 0.2 G and is substantially constant.

8. The dental device of claim 1, wherein the motor is configured to oscillate between accelerations.

9. The dental device of claim 1, wherein the mouthpiece has a U-shaped so as to extend over a plurality of a patient's teeth.

10. The dental device of claim 1, wherein the mouthpiece includes a biteplate configured to sit against occlusal surfaces of a patient's teeth and an extension configured to connect to a base.

11. A method for accelerating orthodontic treatment comprising using the device of claim 1.

12. The method of claim 11 wherein the using step is performed for 5 minutes daily for a plurality of days.

13. A method for seating an orthodontic aligner comprising using the device of claim 1.

14. The method of claim 13 wherein the using step is performed for 5 minutes daily for a plurality of days.

15. A method for treating oral discomfort comprising using the device of claim 1.

16. The method of claim 15 wherein the using step is performed for 5 minutes daily for a plurality of days.

17. A method for stimulating bone growth comprising using the device of claim 1.

18. The method of claim 17 wherein the using step is performed for 5 minutes daily for a plurality of days.
